(19) United States
(12) Patent Application Publication
(10) Pub. No.: US 2006/0036184 A1
(43) Pub. Date: Feb. 16, 2006

(54) Calibration system and method for pressure monitoring

(76) Inventors: Nathan J. Tenzer, Irvine, CA (US); Morgan T. McKeown, Irvine, CA (US)

Correspondence Address: Theodore P. Lopez;Edwards Lifesciences LLC, One Edwards Way, Irvine, CA, 92614, US

(21) Appl. No.: 11/202,601

(22) Filed: Aug. 12, 2005

Related U.S. Application Data
(60) Provisional application No. 60/601,081, filed on Aug. 12, 2004.

(51) Int. Cl. A61B 5/02

(52) U.S. Cl. 600/485

(57) ABSTRACT

A calibration system for pressure monitoring including a sensor positioned at a sensor location on or in a patient's body, a first pressure transducer positioned at a reference location remote from the sensor location to receive a signal from the sensor and to generate a first pressure signal, a calibration device positioned along a plane that is substantially coincident with a chamber or cavity (e.g., a heart chamber) of the patient to measure a reference pressure signal that represents a difference in pressure between the position of the calibration device and the reference location, a second pressure transducer positioned at the reference location remote from the sensor location to receive the reference pressure signal from the calibration device and to generate a calibration pressure signal, and an electronic device to produce an actual pressure signal using the first and calibration pressure signals.

### CROSS-REFERENCE TO RELATED APPLICATIONS

This application claims the benefit under 35 U.S.C. § 119(e) of U.S. patent application Ser. No. 60/601,081, filed Aug. 12, 2004, for “Auto-Zeroing, Auto-Leveling System for Pressure Monitoring,” which is hereby incorporated by reference.

### FIELD OF THE INVENTION

The invention relates generally to the field of pressure monitoring. More particularly, the invention relates to a calibration system and method for pressure monitoring.

### DESCRIPTION OF THE RELATED ART

Physiological pressures (e.g., blood pressure) of a human body can be monitored from different locations on or in the human body. The monitoring can be performed invasively and non-invasively. For example, the monitored pressure can be brachial pressure, central venous pressure, femoral pressure, intracranial pressure, pulmonary artery pressure, radial pressure, right heart pressure, intrauterine pressure, intra-abdominal pressure, etc. These pressures can also be combined with other data to produce further parameters (e.g. cardiac output) which are useful in patient care. One device for monitoring pressure is a pressure transducer (e.g., a sensor attached invasively to the patient via a fluid filled catheter). In order for pressure monitoring to be accurate, the pressure transducer should be at the same vertical level as the body cavity being measured. For example, in order for cardiac pressure monitoring to be accurate, the sensor should be level with the right atrium of the patient. Specifically, if the patient is lying flat, the pressure transducer should be aligned with the phlebostatic axis, determined by the intersection of the midaxillary line and the fourth intercostal space of the patient. If misalignment occurs for any reason (e.g., if the patient bed moves up or down, or if the patient sits up), then the pressure transducer must be recalibrated (i.e. realigned) with the height of the patient's heart in order for the pressure measurements to be accurate.

Current methods of calibrating (i.e. leveling) the pressure transducers include (1) using a carpenter's level to horizontally level the pressure transducer with the heart of the patient, (2) visually estimating the level of the pressure transducer and the heart of the patient, and (3) using a laser pointer to horizontally level the pressure transducer with the heart of the patient. Each of these processes have limitations in achieving and/or maintaining an accurate and consistent height alignment between the pressure sensors and the patient's heart. Other methods of calibrating the pressure transducers for the correct height include directly attaching the pressure transducer to the patient's chest, or attaching the pressure transducer to the patient's bed. These methods introduce limitations in being able to physically access the pressure transducers and the corresponding fluid lines for other purposes, such as flushing the fluid lines or drawing blood samples.

Thus, it should be appreciated that there is a need for accurately monitoring the pressure of a patient (1) without having to re-level the system when the patient changes height or position and/or at regular intervals, and (2) without compromising accessibility to the pressure transducers. The invention fulfills these needs as well as others.

### BRIEF DESCRIPTION OF THE DRAWINGS

FIG. 1 is a front view of a calibration system that may be used with existing pressure monitoring lines according to one embodiment of the invention.

FIG. 2 is a side view of a patient being monitored where the patient's heart is level with the transducers according to one embodiment of the invention.

FIG. 3 is a side view of a patient being monitored where the patient's heart is not level with the transducers according to one embodiment of the invention.

FIG. 4 is a flow chart illustrating a method of pressure monitoring according to one embodiment of the invention.

### SUMMARY OF THE INVENTION

One embodiment of the invention provides a calibration system for pressure monitoring including a sensor positioned at a sensor location on or in a patient's body, a first pressure transducer positioned at a reference location remote from the sensor location to receive a signal from the sensor and to generate a first pressure signal, a calibration device or array of devices positioned along a plane that is substantially coincident with a chamber or cavity (e.g., a heart chamber) of the patient to measure a reference pressure signal that represents a difference in pressure between the position of the calibration device and the reference location, a second pressure transducer positioned at the reference location remote from the sensor location to receive the reference pressure signal from the calibration device and to generate a calibration pressure signal, and an electronic device to produce an actual pressure signal using the first and calibration pressure signals.

One embodiment of the invention provides a method of pressure monitoring including receiving a signal from a sensor, generating a pressure signal using the signal, receiving a reference pressure signal from a calibration device, generating a calibration pressure signal using the reference pressure signal and producing an actual pressure signal using the pressure signal and the calibration pressure signal.

### DETAILED DESCRIPTION

Methods and systems that implement the embodiments of the various features of the invention will now be described with reference to the drawings. The drawings and the associated descriptions are provided to illustrate embodiments of the invention and not to limit the scope of the invention. Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment. Throughout the drawings, reference numbers are re-used to indicate correspondence between referenced elements. In addition, the first digit of each reference number indicates the figure in which the element first appears.

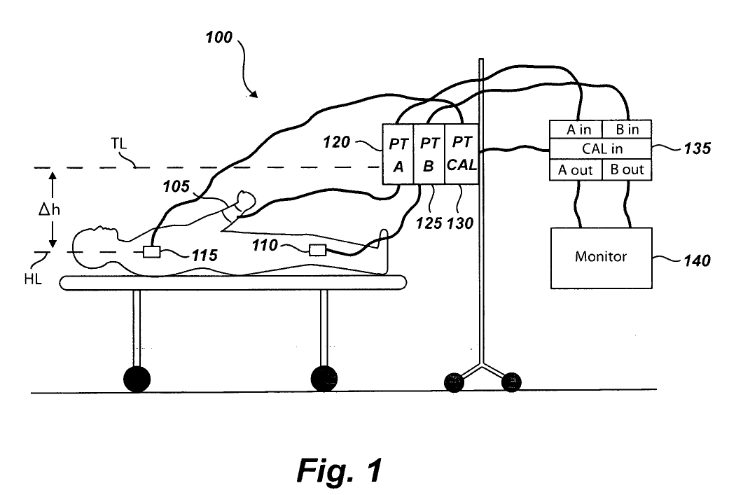

FIG. 1 illustrates a front view of a calibration system 100 that may be used with existing pressure monitoring methods (e.g., pressure transducers, IV bags, tubing, etc.). The calibration system 100 may include a first sensor 105, a second sensor 110, a calibration device 115 (e.g., a static fluid column attached to the patient) and corresponding first, second and calibration pressure transducers 120, 125 and 130. The calibration system 100 may include one or more sensors and one or more pressure transducers. In one embodiment, the second sensor 110 and the second pressure transducer 125 are optional. The connection or link between the sensor and the pressure transducer may be physical (e.g., a fluid column or line), electrical (e.g., wired), wireless, infrared, optical or any other communications medium.

The first and second sensors 105 and 110 may be any device capable of measuring, receiving or propagating a signal from a measurement site (e.g., a location on or in the patient's body). For example, the first and second sensors 105 and 110 may be catheters, finger cuffs, fluid columns or lines, invasive pressure devices, non-invasive pressure devices, piezoelectric devices, pneumatic devices, pressure cuffs, or any other device capable of measuring, receiving or propagating a signal from a measurement site. One skilled in the art will understand that the first and second sensors 105 and 110 do not have to be the same type of device.

The sensor location may be a measurement site on or in the patient's body (402). For example, a clinician may want to measure the pulmonary artery pressure by invasively inserting the first sensor 105 (e.g., a catheter) into an artery of the patient. Once inserted, the first sensor 105 may transmit a signal to the first pressure transducer 120, which generates a first pressure signal (S₁) (404 and 406). Similarly, a clinician may want to measure the brachial pressure by non-invasively attaching the second sensor 110 (e.g., a pressure cuff) to the patient's arm. Once attached, the second sensor 110 may transmit a signal to the second pressure transducer 125, which generates a second pressure signal (S₂).

The calibration device 115 (or array of calibration devices) measures or receives a reference signal that represents a difference in pressure between a reference location (e.g., a patient's heart level) and a pressure transducer location (e.g., the vertical level of the pressure transducer) (410). In one embodiment, the calibration device 115 is positioned at the patient's heart level (HL) and is used to compensate for the height difference (Δh) between the height of the patient's heart (i.e., the reference location) and the height of the pressure transducers located on, for example, an IV pole. In one embodiment, the calibration device 115 is connected to the patient via an adhesive material such as tape or glue (408). Alternatively, the calibration device 115 may be attached to the patient's bed when physical attachment to the patient's body is not feasible, for example, when the patient has suffered severe burns to the chest.

The calibration device 115 may be a fluid column affixed to the patient's body along a horizontal plane that is substantially coincident with a chamber or cavity (e.g., a heart chamber) of the patient. Typically, the fluid column is filled with a fluid such as water or saline and is isolated with a hydrophobic barrier (e.g., a filter, a stopcock, etc.) on one end and is attached to the calibration pressure transducer 130 on the other end. The calibration device 115 may also be a highly sensitive altimeter or an electronic vertical positioning device. The calibration device 115 eliminates the need to re-level the calibration system 100 in response to a change in body position of the patient. That is, any movement of the patient in the vertical direction will not require re-leveling of the pressure transducer location to be in alignment with the sensor location.

As shown in FIG. 1, the first, second and calibration pressure transducers 120, 125 and 130 are positioned along the same horizontal line or at the same height. Specifically, the calibration pressure transducer 130 should be positioned along the same line or plane as the first and second pressure transducers 120 and 125. The first pressure transducer 120 generates a first pressure signal (S₁), the second pressure transducer 125 generates a second pressure signal (S₂) and the calibration pressure transducer 130 generates a calibration pressure signal (S_{C}) (406 and 412). These pressure signals (S₁, S₂ and S_{C}) are transmitted to an electronic device 135. The connection or link between the pressure transducer (120, 125 and 130) and the electronic device 135 may be electrical (e.g., wired), wireless, infrared, optical or any other communications medium. If the pressure transducers are all measuring a cavity located in roughly the same proximity (i.e., cavities within the heart), then the calibration signal can compensate for an unlimited number of pressure transducers.

The electronic device 135 receives the pressure signals and produces first and second actual or true pressure signals (S_{1T} and S_{2T}) by offsetting the first and second pressure signals (S₁ and S₂) using the calibration pressure signal (S_{C}) (414). For example, S_{1T}=S₁−S_{C} and S_{2T}=S₂−S_{C}. Hence, the first and second actual or true signals compensate for any changes in body position of the patient. The electronic device 135 may be a differential circuit or a processor (e.g., a microprocessor). The processor may be implemented using hardware, software or combinations thereof. The first and second actual or true pressure signals are transmitted to a patient monitor 140 for display. The connection or link between the electronic device 135 and the patient monitor 140 may be wired, wireless, infrared, optical or any other communications medium. In one embodiment, the patient monitor 140 can be part of the electronic device 135. In one embodiment, the electronic device 135 can be located at the pressure transducer location or part of any of the pressure transducers (i.e., 120, 125 and/or 130).

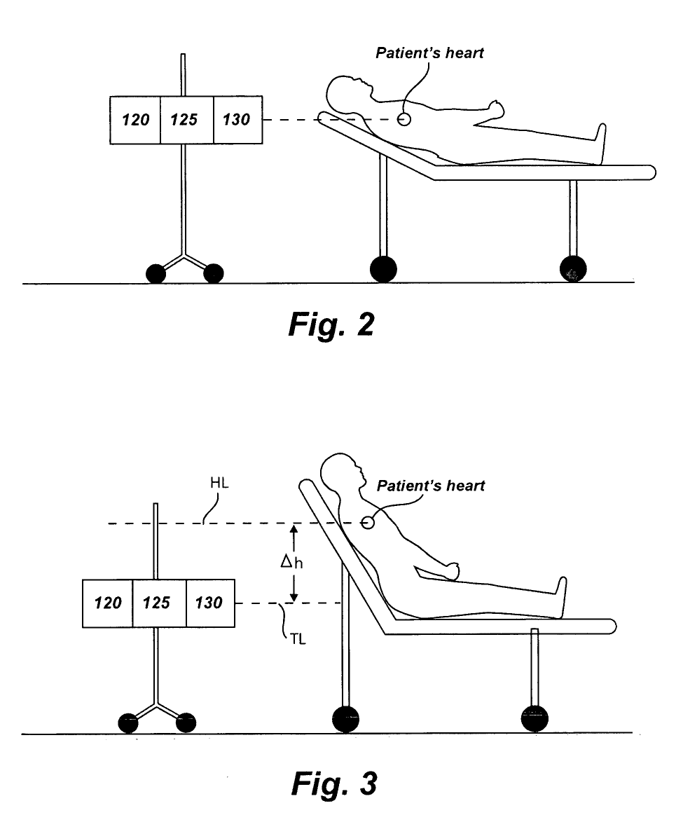

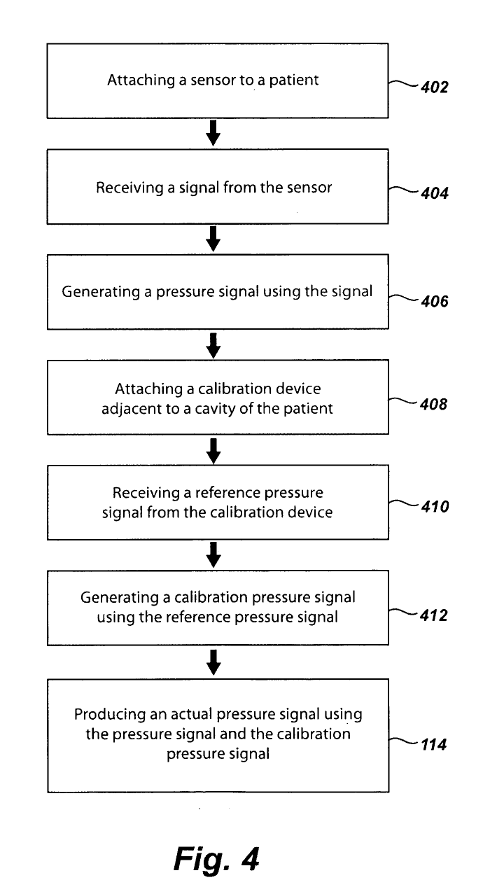

FIG. 2 is a side view of a patient being monitored where the patient's heart is level with the transducers. The patient's heart level is designated as HL and the reference level or transducer level is designated as TL. The patient is lying in bed and being invasively monitored for blood pressure. The patient's heart is level with the transducers. If the patient's body position changes, as shown in FIG. 3, the patient's heart is no longer level with the transducers. Referring to FIG. 3, the calibration for the blood pressure is no longer valid because of the height discrepancy between the height of the patient's heart and the height of the transducers on the IV pole. All the transducers would have to be adjusted for the patient's heart level and then possibly re-zeroed. Using the calibration system 100, no changes to the height of the transducers would need to be made. The calibration device 115 attached at the heart level allows the electronic device 135 to measure the offset to pressures and produce first and second actual or true pressure signals (S_{1T} and S_{2T}) by offsetting the first and second pressure signals (S₁ and S₂) using the calibration pressure signal (S_{C}).

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described preferred embodiment can be configured without departing from the scope and spirit of the invention. Therefore, it is to be understood that, within the scope of the appended claims, the invention may be practiced other than as specifically described herein.

What is claimed is:

1. A calibration system for pressure monitoring comprising:
a sensor positioned at a sensor location on or in a patient's body;
a first pressure transducer positioned at a reference location remote from the sensor location to receive a signal from the sensor and to generate a first pressure signal;
a calibration device positioned along a plane that is substantially coincident with a cavity of the patient to measure a reference pressure signal that represents a difference in pressure between the position of the calibration device and the reference location;
a second pressure transducer positioned at the reference location remote from the sensor location to receive the reference pressure signal from the calibration device and to generate a calibration pressure signal; and
an electronic device to produce an actual pressure signal using the first and calibration pressure signals.

2. The calibration system of claim 1 wherein the sensor is selected from a group consisting of a catheter, a finger cuff, a fluid column, an invasive pressure device, a non-invasive pressure device, a piezoelectric device, a pneumatic device, and a pressure cuff.

3. The calibration system of claim 1 wherein the calibration device is a fluid column.

4. The calibration system of claim 3 wherein the fluid column has a first end that is isolated with a hydrophobic barrier and has a second end that is coupled to the second pressure transducer.

5. The calibration system of claim 1 wherein the first pressure transducer and the second pressure transducer are positioned along the same horizontal plane.

6. The calibration system of claim I wherein the electronic device is a differential circuit.

7. The calibration system of claim I wherein the electronic device is a processor.

8. The calibration system of claim 1 further comprising a monitor to display the actual pressure signal.

9. The calibration system of claim 1 wherein the cavity is a heart cavity or chamber.

10. A calibration system for pressure monitoring comprising:
a sensor positioned at a sensor location on or in a patient's body;
a pressure transducer positioned at a reference location remote from the sensor location to receive a signal from the sensor and to generate a pressure signal;
a fluid column positioned adjacent to a patient's heart to measure a reference pressure signal that represents a difference in pressure between the position of the fluid column and the reference location;
a calibration pressure transducer positioned at the reference location remote from the sensor location to receive the reference pressure signal from the fluid column and to generate a calibration pressure signal; and
an electronic device to produce an actual pressure signal using the pressure signal and the calibration pressure signal.

11. The calibration system of claim 10 wherein the sensor is selected from a group consisting of a catheter, a finger cuff, a fluid column, an invasive pressure device, a non-invasive pressure device, a piezoelectric device, a pneumatic device, and a pressure cuff.

12. The calibration system of claim 10 wherein the fluid column has a first end that is isolated with a hydrophobic barrier and has a second end that is coupled to the calibration pressure transducer.

13. The calibration system of claim 10 wherein the pressure transducer and the calibration pressure transducer are positioned along the same horizontal plane.

14. The calibration system of claim 10 wherein the electronic device is a differential circuit.

15. The calibration system of claim 10 wherein the electronic device is a processor.

16. A method of pressure monitoring comprising:
receiving a signal from a sensor;
generating a pressure signal using the signal;
receiving a reference pressure signal from a calibration device;
generating a calibration pressure signal using the reference pressure signal; and
producing an actual pressure signal using the pressure signal and the calibration pressure signal.

17. The method of claim 16 further comprising attaching the sensor to a patient.

18. The method of claim 17 further comprising attaching the calibration device adjacent to the heart of the patient.

19. The method of claim 16 wherein the sensor is selected from a group consisting of a catheter, a finger cuff, a fluid column, an invasive pressure device, a non-invasive pressure device, a piezoelectric device, a pneumatic device, and a pressure cuff.

20. The method of claim 16 wherein the calibration device is a fluid column.
